(19) United States
(12) Patent Application Publication
(10) Pub. No.: US 2025/0288032 A1
(43) Pub. Date: Sep. 18, 2025

(54) BUFFERING SHEET FOR GOGGLES

(71) Applicant: INOAC CORPORATION, Nagoya-shi, Aichi (JP)

(72) Inventor: Hiroshi NAKAO, Ukiha-shi, Fukuoka (JP)

(21) Appl. No.: 18/860,691

(22) PCT Filed: Apr. 19, 2023
(86) PCT No.: PCT/JP2023/015623

§ 371 Date: Oct. 28, 2024

(30) Foreign Application Priority Data
Apr. 29, 2022 (JP) 2022-075555

(51) Int. Cl. A41D 13/11; A41D 13/015

(52) U.S. Cl. CPC A41D 13/11; A41D 13/015

(57) ABSTRACT

A buffering sheet for goggles, comprising: a buffering portion that is disposed on a face; and an attachment portion that is used for attachment to a head, wherein the buffering portion is formed by a sheet having a resin foam layer, and an elongation percentage of the sheet is 50% or more.

### TECHNICAL FIELD

The present invention relates to a buffering sheet for goggles used when a head-mounted display, VR goggles, or the like is worn.

### BACKGROUND ART

For example, there are goggles for VR goggles and head-mounted displays. These goggles are worn on the head with a pad of an image display unit in contact with the periphery of the eyes on the face. In a case in which goggles are shared, it is not hygienically preferable to apply the pad directly to the face. Accordingly, it has been proposed that an antifouling sheet made by a nonwoven fabric is attached to the face in advance so that the pad of goggles does not come into contact with the face (see, for example, Patent Document 1).

### CITATION LIST

### Patent Document

Patent Document 1: Japanese Patent Application Laid-Open (JP-A) No. 2016-170196

### SUMMARY OF INVENTION

### Technical Problem

The sheet of Patent Document 1 does not take into consideration elongation of the sheet in a plane direction, and thus the sheet has poor fitting to unevenness of the face.

The present disclosure has been proposed in view of the problems related to the conventional technique in order to suitably solve these problems, and an object of the present disclosure is to provide a buffering sheet for goggles excellent in fitting to the face.

### Solution to Problem

One aspect of a buffering sheet for goggles according to the disclosure is
a buffering sheet for goggles including:
a buffering portion that is disposed on a face; and
an attachment portion that is used for attachment to a head, wherein
the buffering portion is formed by a sheet having a resin foam layer, and
an elongation percentage of the sheet is 50% or more.

In one aspect of the buffering sheet for goggles according to the disclosure, in addition to the above matters, the attachment portion may include the sheet.

In one aspect of the buffering sheet for goggles according to the disclosure, in addition to the above matters, an upper edge of the buffering portion may have a convex shape bulging upward.

In one aspect of the buffering sheet for goggles according to the disclosure, in addition to the above matters, a thickness of the sheet may be 1.0 mm or more.

In one aspect of the buffering sheet for goggles according to the disclosure, in addition to the above matters, 25% ILD hardness of the sheet may be 40 N or more.

### Advantageous Effects of Invention

A buffering sheet for goggles according to the disclosure is excellent in fitting to the face.

### BRIEF DESCRIPTION OF DRAWINGS

FIG. 1 is a front view illustrating a buffering sheet for goggles according to an embodiment of the present invention.

FIG. 2 is a plan view illustrating the buffering sheet for goggles of the embodiment.

FIG. 3 is a side view illustrating the buffering sheet for goggles of the embodiment.

FIG. 4 is a transverse cross-sectional view of the buffering sheet for goggles of the embodiment. It is taken along line X-X of FIG. 1.

FIG. 5 is a schematic perspective view illustrating a state in which the buffering sheet for goggles of the embodiment is worn.

FIG. 6 is a side view illustrating a state in which the buffering sheet for goggles of the embodiment is worn.

FIG. 7 is a front view illustrating a modification of an eye opening portion.

FIG. 8 is a transverse cross-sectional view of a buffering sheet for goggles of the modification. It is taken along line Y-Y of FIG. 7.

FIG. 9 is a schematic perspective view illustrating a state in which the buffering sheet for goggles of the modification is worn.

FIG. 10 is a side view illustrating a state in which the buffering sheet for goggles of the modification is worn.

### DESCRIPTION OF EMBODIMENTS

Next, a buffering sheet for goggles according to the disclosure will be described below with reference to the accompanying drawings by way of preferred embodiments.

### Embodiment

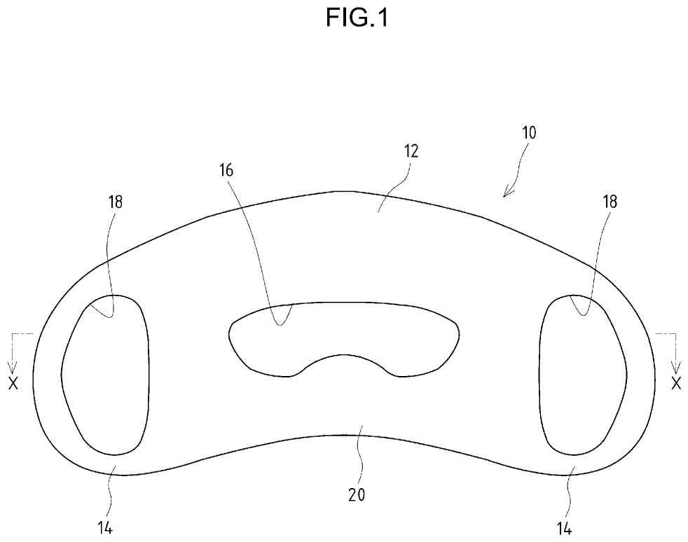

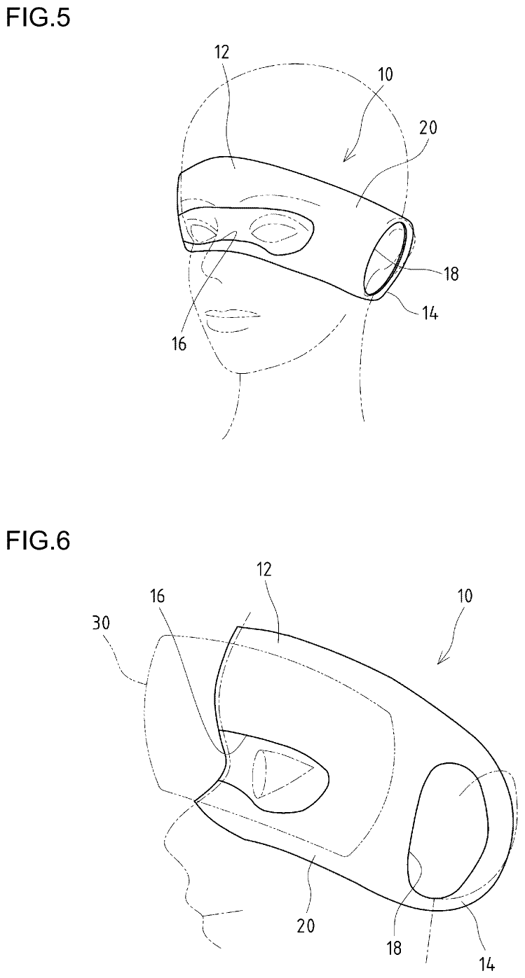

As illustrated in FIG. 1, a buffering sheet for goggles (hereinafter, the buffering sheet is simply referred to as a buffering sheet) 10 according to an embodiment includes a buffering portion 12 that is disposed on the face. The buffering sheet 10 includes an attachment portion 14 that is used for attaching to the head. As illustrated in FIG. 5, the buffering sheet 10 is used by disposing the buffering portion 12 on the face and attaching the attachment portion 14 to the head of the user. As illustrated in FIG. 6, when the user wears goggles 30 such as a head-mounted display or VR goggles, the buffering sheet 10 receives a rear edge portion of the goggles 30 by the buffering portion 12.

The buffering portion 12 is formed in accordance with a portion of the goggles 30 that contacts the face. As illustrated in FIGS. 5 and 6, the buffering portion 12 is formed in a size that can be disposed around the user's eyes. In the embodiment, an upper portion of the buffering portion 12 covers the forehead of the user, a lower portion of the buffering portion 12 covers an area from the nose root to the cheekbones of the user, and a side portion of the buffering portion 12 covers an area from the outer corner of each eye to the ear of the user.

As illustrated in FIG. 1, the upper portion of the buffering portion 12 has a convex shape bulging upward. With such a shape, the upper portion of the buffering portion 12 is disposed on the forehead, and the attached buffering sheet 10 can be hardly displaced downward. In addition, when the buffering sheet 10 is attached, the buffering sheet 10 can be stretched at the upper portion of the buffering portion 12, and the buffering sheet can be hardly displaced downward. The lower portion of the buffering portion 12 has a recessed shape recessed upward. The buffering portion 12 has a curved shape in which side portions are lower than a central portion in a left-right direction. Even with such a shape, the upper portion of the buffering portion 12 is disposed on the forehead, and the attached buffering sheet 10 can be hardly displaced downward. In the embodiment, the upper edge and the lower edge of the buffering portion 12 are parallel curves.

As illustrated in FIG. 1, the buffering portion 12 is provided with an eye opening portion 16 in which the user's eyes are arranged. As illustrated in FIG. 4, the eye opening portion 16 of the embodiment is formed in a hole shape penetrating the buffering portion 12. The shape of the eye opening portion 16 may be a circle, an ellipse, a shape derived from an ellipse, a polygon such as a quadrangle, or the like, and may be configured singly or in any combination thereof. The eye opening portion 16 is provided at one location at a center of the buffering portion 12 in the left-right direction. For example, as in the eye opening portion 16 of the embodiment, if the hole is horizontally long corresponding to both eyes, the buffering sheet 10 can be worn while wearing the glasses. In the eye opening portion 16, the vertical width of the central portion in the left-right direction corresponding to a space between the left and right eyes is formed to be narrower than a vertical width of the left and right side portions facing the positions of the eyes.

As illustrated in FIG. 1, the attachment portions 14 are provided on both side portions of the buffering portion 12. The attachment portion 14 is provided to be shifted downward from the buffering portion 12. With such a shape, it is possible to stretch the upper portion of the buffering portion 12 in contact with the forehead and to make the attached buffering sheet 10 can be hardly displaced downward. The attachment portion 14 of the embodiment is connected to a side portion of the buffering portion 12 extending obliquely downward from the central portion in the left-right direction toward the side, and extends obliquely downward so as to be inclined obliquely downward. In this way, the attachment portion 14 naturally faces the ear located slightly below the eye position, so that the attachment portion can be easily attached.

As illustrated in FIG. 1, the attachment portion 14 of the embodiment has an ear hooking hole 18 for hooking on the user's ear. The shape of the ear hooking hole 18 is preferably a vertically long ellipse or a shape derived from the ellipse. The width of an opening edge of the ear hooking hole 18 in the attachment portion 14 is preferably wider than a rubber string, and can be set to, for example, from 5 mm to 20 mm. Note that the ear hooking hole 18 is disposed at a position biased downward in the vertical direction of the attachment portion 14. The buffering sheet 10 is attached by inserting the ear through the ear hooking hole 18, and hooking the opening edge of the ear hooking hole 18 in the attachment portion 14 to a base of the ear.

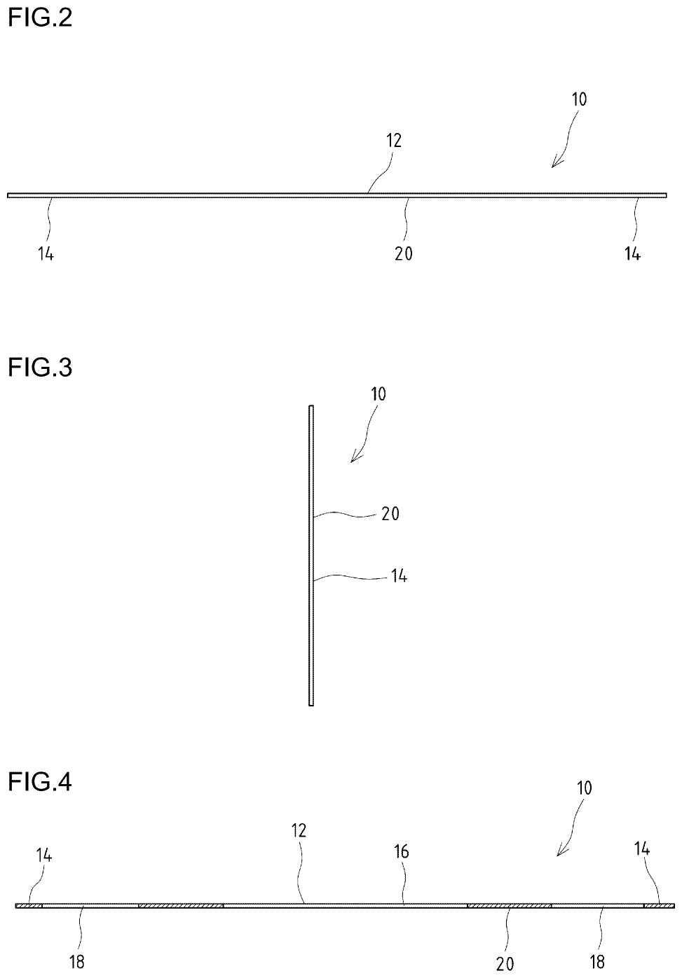

As illustrated in FIGS. 2 to 4, the buffering portion 12 is formed by a sheet 20 having a resin foam layer. By forming the buffering portion 12 with the sheet 20 having the resin foam layer as described above, the buffering portion is easily attached and detached. The buffering portion 12 follows the unevenness of the face during wearing, and is less likely to be displaced. The buffering portion 12 stretches and follows the movement of the face such as a change in expression, and thus discomfort and a load on the user can be reduced.

The attachment portion 14 is formed by a sheet 20 having a resin foam layer. By forming the attachment portion 14 with the sheet 20 having the resin foam layer as described above, the attachment portion is easily attached and detached. The attachment portion 14 fits the head and is less likely to be displaced. The attachment portion 14 stretches and follows the movement of the face such as a change in expression, and thus discomfort and a load on the user can be reduced. In the embodiment, the buffering portion 12 and the right and left attachment portions 14 are formed by one sheet 20 (see FIGS. 2 to 4). The sheet 20 may have a multilayer structure having, for example, a fabric layer in addition to the resin foam layer, but the sheet 20 of the embodiment is constituted only by the resin foam layer.

The eye opening portion 16 and the ear hooking hole 18 may have a notched shape partially opened to an outer edge of the sheet 20, but it is preferable to have an independent hole shape surrounded by the sheet 20. In this way, moderate stretching and contraction of the buffering portion 12 and the attachment portion 14 are exhibited in a well-balanced manner, and the buffering portion and the attachment portion can be fitted and hardly displaced at the time of attachment.

Examples of the resin foam layer include polyurethane foams, polyolefin foams such as polyethylene (PE) foams, ethylene vinyl acetate copolymer (WVA) foams, and polypropylene (PP) foams, and rubber sponges. As the resin foam, a soft foam that is relatively soft and has resiliency is preferable. Among them, a polyurethane foam is preferable from the viewpoint of cost and the like. The polyurethane foam may be either polyether-based or polyester-based. For example, in a case in which a polyether-based polyurethane foam is used, water resistance (moisture and heat aging resistance) can be improved. The polyurethane foam may be formed by slab molding, an extraction method, or other molding methods.

For the resin expanded article layer, it is preferable to use a membrane-removed product in which a cell wall (film) between adjacent bubbles is removed to form only a skeleton, or a continuous structure or semi-continuous structure product in which holes are formed in each cell wall (film), because stickiness due to sweat is prevented at the time of wearing, and stuffiness is less likely to occur. The membrane-removed product can be obtained by a known film removal treatment, for example, a method of dissolving the cell wall with a solvent, a method of breaking the cell wall by explosion, or the like. When the air bubbles are exposed on a surface of the sheet 20, the buffering portion 12 and the attachment portion 14 can be hardly displaced due to unevenness of the surface due to the air bubbles.

A thickness of the sheet 20 is preferably 0.8 mm or more, more preferably 1.0 mm or more, and still more preferably 1.2 mm or more. With the buffering portion 12 formed by such a sheet 20, force applied from the goggles 30 can be received and dispersed by the buffering portion 12. In a case in which the attachment portion 14 is formed by the sheet 20, it is possible to make it difficult for the attachment portion to be damaged. The thickness of the sheet 20 is preferably 10 mm or less, more preferably 5 mm or less, and still more preferably 3 mm or less. With the buffering portion 12 formed by such a sheet 20, it is difficult to break and bulkiness can be suppressed. In a case in which the attachment portion 14 is formed by the sheet 20, the attachment portion is hardly damaged, and bulkiness can be suppressed.

The sheet 20 has extensibility in a plane direction thereof. With the buffering portion 12 formed by such a sheet 20, the buffering portion can be fitted to the face, and a load at the time of wearing can be reduced. An elongation percentage of the sheet 20 is preferably 50% or more, more preferably 70% or more, still more preferably 80% or more, and particularly preferably 90% or more. With the buffering portion 12 formed by such a sheet 20, the buffering portion can be fitted to the face and can be easily worn. In a case in which the attachment portion 14 is formed by the sheet 20, the buffering sheet 10 can be easily mounted. The elongation percentage of the sheet 20 is preferably 500% or less, more preferably 450% or less, and still more preferably 400% or less. With the buffering portion 12 formed by such a sheet 20, the buffering portion is less likely to be damaged, can be fitted to the face, and can reduce the load at the time of wearing. In a case in which the attachment portion 14 is formed by the sheet 20, the attachment portion 14 is hardly damaged, and the load at the time of wearing can be reduced. The elongation percentage is measured based on JIS K 6400-5: 2004 Dumbbell No. 2.

The sheet 20 preferably has a cushioning property of being elastically deformed in the thickness direction thereof. The hardness (25% ILD) of the sheet 20 is preferably 40 N or more, more preferably 80 N or more, and still more preferably 100 N or more. With the buffering portion 12 formed by such a sheet 20, force applied from the goggles 30 can be received and dispersed by the buffering portion 12. In a case in which the attachment portion 14 is formed by the sheet 20, it is possible to make it difficult for the attachment portion to be damaged. The hardness (25% ILD) of the sheet 20 is preferably 300 N or less, more preferably 250 N or less, and still more preferably 200 N or less. With the buffering portion 12 formed by such a sheet 20, the buffering portion can be fitted to the face and can be easily worn. In a case in which the attachment portion 14 is formed by the sheet 20, the buffering sheet 10 can be easily mounted. The hardness described above is measured by a hardness test method in 25% compression (method D) using a pressing plate of a flat disk having a diameter of 200 mm according to JIS K 6400-2: 2012.

Density of the sheet 20 is preferably 10 kg/m³ or more, more preferably 13 kg/m³ or more, and still more preferably 20 kg/m³ or more. With the buffering portion 12 formed by such a sheet 20, the buffering portion is less likely to be damaged, can be fitted to the face, and can reduce the load at the time of wearing. In a case in which the attachment portion 14 is formed by the sheet 20, it is possible to make it difficult for the attachment portion to be damaged. The density of the sheet 20 is preferably 150 kg/m³ or less, more preferably 120 kg/m³ or less, and still more preferably 100 kg/m³ or less. With the buffering portion 12 formed by such a sheet 20, the weight can be reduced, and the load at the time of wearing can be reduced. In a case in which the attachment portion 14 is formed by the sheet 20, the weight can be reduced, and the load at the time of wearing can be reduced. The density is measured based on JIS K 7222: 2005.

The cell size of the sheet 20 is preferably 20 cells/25 mm or more, more preferably 30 cells/25 mm or more, and still more preferably 40 cells/25 mm or more. With the buffering portion 12 formed by such a sheet 20, the weight can be reduced, and the load at the time of wearing can be reduced. In a case in which the attachment portion 14 is formed by the sheet 20, the weight can be reduced, and the load at the time of wearing can be reduced. The cell size of the sheet 20 is preferably 100 cells/25 mm or less, more preferably 90 cells/25 mm or less, and still more preferably 80 cells/25 mm or less. With the buffering portion 12 formed by such a sheet 20, the buffering portion is less likely to be damaged, can be fitted to the face, and can reduce the load at the time of wearing. In a case in which the attachment portion 14 is formed by the sheet 20, it is possible to make it difficult for the attachment portion to be damaged. The cell size is measured based on JIS K 6400-1: 2004 Annex 1 (Reference).

The sheet 20 preferably has air permeability. The air permeability of the sheet 20 is preferably 60 cm³/(cm²·sec) or more, more preferably 100 cm³/(cm²·sec) or more, and still more preferably 150 cm³/(cm²·sec) or more. With the buffering portion 12 formed by such a sheet 20, stuffiness is less likely to occur, and a load at the time of wearing can be reduced. In a case in which the attachment portion 14 is formed by the sheet 20, stuffiness is less likely to occur, and a load at the time of wearing can be reduced. The above-mentioned air permeability is a value measured using a method described in JIS L 1096-7: 2010 “Fabric test method for woven and knitted fabrics: Method A (Frazier method)”.

As the sheet 20, it is preferable to use a sheet which is subjected to a treatment for reducing odor derived from a chemical substance such as a foaming agent and is excellent in cleanliness (cleaned product). Examples of the clean treatment include a treatment for removing chemical substances remaining in the resin foam, such as washing the resin foam with water or the like or heating (for example, from 70 to 120° C., from 30 to 120 minutes) to deodorize the resin foam.

The buffering sheet 10 of the embodiment can be easily formed, for example, by punching the sheet 20 having a required thickness with a Thomson blade or the like.

In the buffering sheet 10, the buffering portion 12 is disposed on the face by matching the eye opening portion 16 with the left and right eyes and hooking the ear hooking holes 18 of the attachment portions 14 on the ears. With the buffering sheet 10, since the goggles 30 are received by the buffering portion 12 made by the sheet 20 having the resin foam layer, the force applied from the goggles 30 can be dispersed to reduce a burden on the user.

The buffering portion 12 has appropriate stretchability, and thus it is easy to attach and detach. The buffering portion 12 follows the unevenness of the face during wearing, and is less likely to be displaced. The buffering portion 12 stretches and follows the movement of the face such as a change in expression, and thus discomfort and a load on the user can be reduced.

The buffering portion 12 has a buffering property with an appropriate hardness (25% ILD), and thus it is possible to disperse the force applied from the goggles 30 when receiving the goggles 30 and to reduce a burden on the user. The goggles deform following the unevenness of the face and the shape of the rear edge of the goggles 30, and thus the goggles can be widely used for the goggles 30 having different face shapes and shapes. A gap is less likely to occur between the goggles 30 and the buffering sheet 10, and thus the shielding property capable of preventing light leakage and the like can be improved.

Because the buffering portion 12 has an appropriate thickness, the force applied from the goggles 30 when receiving the goggles 30 can be dispersed, and the burden on the user can be reduced. Further, it is possible to make it difficult to break.

Because the upper portion of the buffering portion 12 has an upward convex shape, it is possible to avoid partial extreme extension of the buffering portion 12 along the three-dimensional structure of the face. In addition, it is possible to obtain a cushioning effect on the face around the eyes and an effect of causing a certain stretch in the forehead so as not to be displaced downward when worn.

The attachment portion 14 has appropriate stretchability, and thus it is easy to attach and detach. It is possible to reduce a sense of discomfort and a load on the user when worn. The attachment portion 14 has a cushioning property with an appropriate hardness (25% ILD), and thus the burden on the user can be reduced. Since the attachment portion 14 has an appropriate thickness, the force can be dispersed when the attachment portion is attached to the head (ear), and the burden on the user can be reduced. Further, it is possible to make it difficult to break.

### (Test)

A buffering sheet of a test example including a sheet made by a flexible polyurethane foam and a buffering sheet of a reference example including a sheet made by a nonwoven fabric were manufactured in the same shape, and their mountability was evaluated. The elongation percentage and hardness of each sheet are as illustrated in Tables 1 and 2 below. The sheet made by nonwoven fabric has no cushioning property, and 25% ILD cannot be measured. For the wearability, the buffering sheet was actually worn, and three items of (1) ease of wearing, (2) fit, and (3) load at the time of wearing were evaluated. A case in which one or more of the items were better than that in the reference example was evaluated as “A”, a case in which two or more of the items were better than that in the reference example was evaluated as “AA”, and a case in which one or more of the items were worse than that in the reference example was evaluated as “C”.

TABLE 1

| | Test | Test | Test | Test | Test | Test | Test | Reference |
|---|---|---|---|---|---|---|---|---|
| | Example 1 | Example 2 | Example 3 | Example 4 | Example 5 | Example 6 | Example 7 | Example |
| Thickness | 1.0 | 1.0 | 1.0 | 1.0 | 1.0 | 1.0 | 1.0 | 0.5 |
| (mm) | | | | | | | | |
| Elongation | 50 | 70 | 80 | 30 | 400 | 450 | 500 | 20 |
| percentage | | | | | | | | |
| (%) | | | | | | | | |
| Evaluation | A | AA | AA | AA | AA | AA | AA | — |

TABLE 2

| | Test | Test | Test | Test | Test | Test | Reference |
|---|---|---|---|---|---|---|---|
| | Example 8 | Example 9 | Example 10 | Example 11 | Example 12 | Example 13 | Example |
| Thickness | 1.0 | 1.0 | 1.0 | 1.0 | 1.0 | 1.0 | 0.5 |
| 25% ILD | 40 | 80 | 100 | 200 | 250 | 300 | — |
| (N) | | | | | | | |
| Evaluation | A | AA | AA | AA | AA | AA | — |

According to Table 1, it is found that when the sheet is in the range of the elongation percentage of Test Examples 1 to 7, favorable wearability can be obtained. According to Table 2, it is found that when the sheet is in the hardness range of Test Examples 8 to 13, favorable wearability can be obtained.

### Modification

The invention is not limited to the above-described matters, and for example, the following may be performed. Note that the present invention is not limited only to the specific description of the embodiment and the following modification.

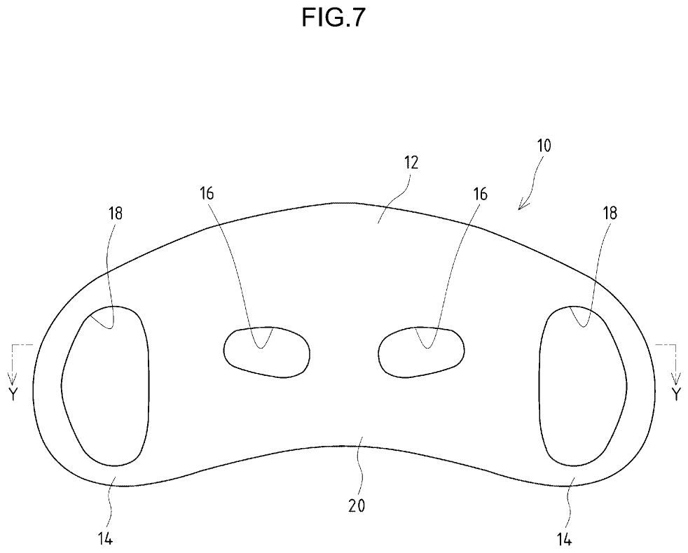

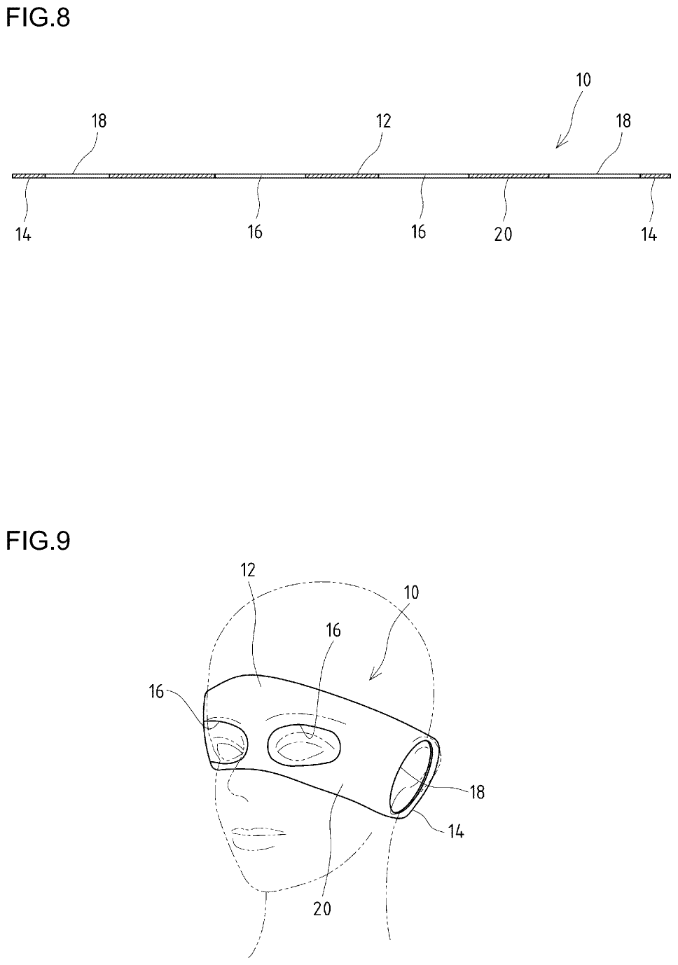

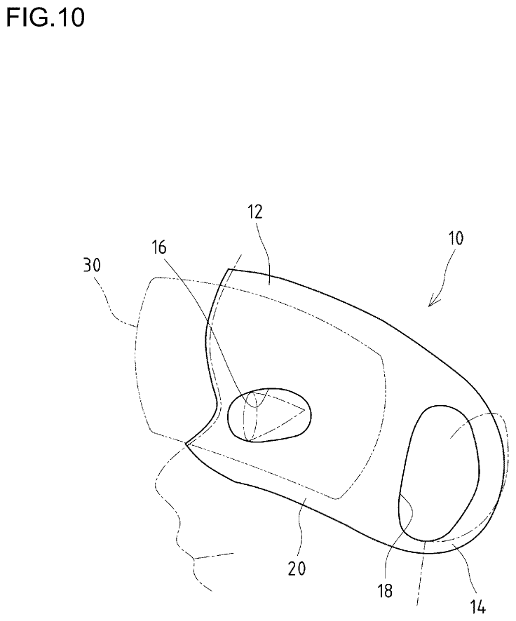

(1) The shape and number of the eye opening portions are not limited to the embodiments. For example, as illustrated in FIGS. 7 to 10, the two eye opening portions 16 may be formed to be separated in the left-right direction. The eye opening portion 16 is a hole penetrating the buffering portion 12. In the modification, the buffering portion 12 and the attachment portion 14 are the same as those in the embodiment. In the buffering sheet 10 of the modification, the left and right eye opening portions 16 are aligned with the left and right eyes, and the attachment portion 14 is attached to the ear, so that the buffering portion 12 is disposed on the face. The buffering sheet 10 of the modification has the same plane and side surface as those of the embodiment. As in the modified example, the independent eye opening portions 16 corresponding to the left and right eyes can cover and protect the nose root portion with the buffering portion 12.

(2) The sheet may include a layer other than the resin foam layer. Examples thereof include a fiber layer in which fibers such as a knitted fabric, a woven fabric, and a nonwoven fabric are formed into a sheet shape, and a coating layer such as a resin. The sheet preferably satisfies the above-mentioned physical properties such as elongation and hardness even in a multilayer structure. The fabric used for the sheet is preferably a knitted fabric, and examples thereof include tricot and circular knitting. The nonwoven fabric is preferably a material that follows elongation, and examples thereof include spun lace. In any configuration, the material is not particularly limited, but may be any of polyester, nylon, rayon, urethane, cotton, synthetic fibers, natural fibers, and the like. In order to secure the elongation of the material, a stretchable material such as a urethane material or a stretch material may be used to facilitate the structural elongation.

(3) The buffering sheet is not limited to the configuration in which the buffering portion and the attachment portion are formed by one sheet, and the buffering portion and the attachment portion may be formed by joining separate sheets. Two or more sheets may be joined to constitute the buffering portion. The attachment portion may be formed by joining two or more sheets.

(4) The attachment portion is not limited to a sheet, and may be, for example, an elastic material having elasticity such as a string or rubber.

(5) The attachment portion is not limited to the configuration in which the ear is hooked to the ear hooking hole, and may be in other attachment modes such as connecting the left and right attachment portions at the back of the head, or fitting a buffering sheet formed in an annular shape by connecting the left and right attachment portions to the head.

The disclosure describes a buffering sheet for goggles including:
a buffering portion that is disposed on a face; and
an attachment portion that is used for attachment to a head, in which
the buffering portion is formed by a sheet having a resin foam layer.

With the buffering sheet for goggles, since the goggles are received by the buffering portion, the force applied from the goggles can be dispersed to reduce the burden on the user.

### REFERENCE SIGNS LIST

10 Buffering sheet for goggles (buffering sheet)
12 Buffering portion
14 Attachment portion
20 Sheet

1. A buffering sheet for goggles, comprising:
a buffering portion that is disposed on a face; and
an attachment portion that is used for attachment to a head,
wherein the buffering portion is formed by a sheet having a resin foam layer, and
an elongation percentage of the sheet is 50% or more.

2. The buffering sheet for goggles according to claim 1,
wherein the attachment portion includes the sheet.

3. The buffering sheet for goggles according to claim 1,
wherein an upper edge of the buffering portion has a convex shape bulging upward.

4. The buffering sheet for goggles according to claim 1,
wherein a thickness of the sheet is 1.0 mm or more.

5. The buffering sheet for goggles according to claim 1,
wherein 25% ILD hardness of the sheet is 40 N or more.

6. The buffering sheet for goggles according to claim 1,
wherein an upper edge of the buffering portion has a convex shape bulging upward, and
a lower edge of the buffering portion has a concave shape recessed upward.

7. The buffering sheet for goggles according to claim 6,
wherein the upper edge and the lower edge of the buffering portion are curves.

8. The buffering sheet for goggles according to claim 7,
wherein the upper edge of the buffering portion and an upper edge of the attachment portion are curves as wholes, and
the lower edge of the buffering portion and a lower edge of the attachment portion are curves as wholes.

9. The buffering sheet for goggles according to claim 1,
wherein the attachment portion extends from the buffering portion so as to be inclined obliquely downward.

10. The buffering sheet for goggles according to claim 1,
wherein the elongation percentage of the sheet is 70% or more, and
25% ILD hardness of the sheet is 80 N or more.
